(19) United States
(12) Patent Application Publication
(10) Pub. No.: US 2026/0013724 A1
(43) Pub. Date: Jan. 15, 2026

(54) WIDE-ANGLE VIEWING SYSTEM FOR AN OPHTHALMIC MICROSCOPE

(71) Applicant: Alcon Inc., Fribourg (CH)

(72) Inventor: Douglas ALLEN, Los Angeles, CA (US)

(21) Appl. No.: 19/266,037

(22) Filed: Jul. 10, 2025

Related U.S. Application Data
(60) Provisional application No. 63/670,675, filed on Jul. 12, 2024.
(60) Provisional application No. 63/670,682, filed on Jul. 12, 2024.

(51) Int. Cl. A61B 3/13; A61B 3/00

(52) U.S. Cl. CPC A61B 3/13; A61B 3/0075

(57) ABSTRACT

Certain embodiments provide an arm assembly for a wide angle viewing system for an ophthalmic microscope. The arm assembly comprises an articulated arm configured to be coupled to a reduction lens module, and a loupe lens turret coupled to the articulated arm. The loupe lens turret comprises a body including a first magnet, and a base including a second magnet. The base is rotationally coupled to the body, and configured to receive a removable loupe lens assembly. The first magnet and the second magnet are configured to generate a first repulsive magnetic force and a second repulsive magnetic force. The first repulsive magnetic force rotates the base in a first direction and holds the base against the body in a first position. The second repulsive magnetic force rotates the base in a second direction and holds the base against the body in a second position.

### CROSS-REFERENCE TO RELATED APPLICATIONS

This application claims priority to and benefit of U.S. Provisional Application No. 63/670,675, filed Jul. 12, 2024, and U.S. Provisional Application No. 63/670,682, filed Jul. 12, 2024, both of which are hereby assigned to the assignee hereof and hereby expressly incorporated by reference in their entirety as if fully set forth below and for all applicable purposes.

### BACKGROUND

The present disclosure relates to ophthalmic surgery. More particularly, the present disclosure relates to a wide-angle viewing system (WAVS) for an ophthalmic microscope.

Ophthalmic microscopes are essential to many ophthalmic surgeries, allowing the surgeon to perform the procedure safely, precisely, and efficiently. Ophthalmic microscopes provide high contrast and detailed imaging of the different regions of the human eye, and may include or support a variety of features, such as advanced visualization, customizable illumination, high-quality imaging at lower illumination levels, instrument connectivity, etc. Adding a WAVS to the ophthalmic microscope generally provides a panoramic or wide-angle view of the surgical field to the surgeon, such as the fundus of the eye.

### SUMMARY

Certain embodiments provide an arm assembly for a wide angle viewing system for an ophthalmic microscope. The arm assembly comprises an articulated arm configured to be coupled to a reduction lens module, and a loupe lens turret coupled to the articulated arm. The loupe lens turret comprises a body including a first magnet, and a base including a second magnet. The base is a base rotationally coupled to the body, and configured to receive a removable loupe lens assembly. The first magnet and the second magnet are configured to generate a first repulsive magnetic force and a second repulsive magnetic force. The first repulsive magnetic force rotates the base in a first direction and holds the base against the body in a first position. The second repulsive magnetic force rotates the base in a second direction and holds the base against the body in a second position.

### BRIEF DESCRIPTION OF THE DRAWINGS

FIGS. 1A and 1B depict a left side view of an example WAVS in a stowed position and a deployed position (respectively), in accordance with embodiments of the present disclosure.

FIG. 2 depicts a perspective view of the example WAVS during a patient procedure, in accordance with embodiments of the present disclosure.

FIG. 3 depicts a perspective view of a portion of a reduction lens module and a portion of an arm assembly of the example WAVS, in accordance with embodiments of the present disclosure.

FIG. 4A depicts a perspective view of an arm assembly of the example WAVS, in accordance with embodiments of the present disclosure.

FIG. 4B depicts a perspective sectional view of the arm assembly depicted in FIG. 4A, in accordance with embodiments of the present disclosure.

FIGS. 5A and 5B depict perspective views of a portion of a loupe lens turret and a loupe lens assembly of the arm assembly depicted in FIG. 4A, in accordance with embodiments of the present disclosure.

FIG. 6 depicts a perspective sectional view of a portion of a loupe lens turret and a portion of a loupe lens assembly of the arm assembly depicted in FIG. 4A, in accordance with embodiments of the present disclosure.

FIGS. 7A and 7B depict a left side view and a left side sectional view (respectively) of a portion of a loupe lens turret and a loupe lens assembly in a procedure position, in accordance with embodiments of the present disclosure.

FIGS. 7C and 7D depict a left side view and a left side sectional view (respectively) of a portion of a loupe lens turret and a loupe lens assembly in a safety position, in accordance with embodiments of the present disclosure.

FIGS. 8A, 8B, 8C depict perspective sectional views of a portion of a loupe lens turret and a loupe lens assembly in a procedure position, an inflection position, and a safety position (respectively), in accordance with embodiments of the present disclosure.

FIG. 9 depicts process an example flow diagram presenting functionality associated with the arm assembly of the example WAVS, in accordance with certain embodiments of the present disclosure.

### DETAILED DESCRIPTION

Generally, a WAVS for an ophthalmic microscope includes a lens system that provides a wide-angle view of the fundus of a patient's eye. The lens system includes one or more lenses that are positioned between the ophthalmic microscope and the patient's cornea. For example, the lens system may include a reduction lens located proximate to the ophthalmic microscope, and a loupe lens located proximate to the patient's cornea. The optical axes of the reduction lens and the loupe lens are aligned with the optical axis of the ophthalmic microscope during the ophthalmic procedure.

The proximity of the loupe lens to the patient's cornea may be adjusted during the focusing procedure of the ophthalmic microscope. However, if the surgeon contacts the patient's cornea with the loupe lens during the focusing procedure, the surgeon must remove the loupe lens from the patient's cornea, clean the loupe lens, and then repeat the focusing procedure.

Certain embodiments of the present disclosure advantageously provide an arm assembly for a WAVS that rotates the loupe lens away from the patient's cornea after contact and without intervention by the surgeon. In certain embodiments, the loupe lens is attached to a rotating base of a loupe lens turret of the arm assembly. In certain embodiments, a stationary magnet is attached to the body of the loupe lens turret, and a rotating magnet is attached to the rotating base. In certain embodiments, the repulsing sides of the stationary and rotating magnets face one another, and are configured to generate a first repulsive magnetic force that causes the loupe lens and the rotating base to rotate in a first direction (such as a positive rotation), and a second repulsive magnetic force that causes the loupe lens and the rotating base to rotate in a second direction (such as a negative rotation).

In certain other embodiments, the attractive (i.e., attracting) sides of the stationary and rotating magnets face one another, and are configured to generate the first repulsive magnetic force that causes the loupe lens and the rotating base to rotate in the first direction, and the second repulsive magnetic force that causes the loupe lens and the rotating base to rotate in the second direction.

In certain embodiments, a procedure position (also know as a first position), the flux centerline of the rotating magnet is situated parallel to and offset from the flux centerline of the stationary magnet, which generates the first repulsive magnetic force that holds the rotating base against a stop on the body of the loupe lens turret.

During the focusing procedure, in certain embodiments, the WAVS moves towards the cornea along a linear path. In certain embodiments, when the loupe lens contacts the cornea, the loupe lens and the rotating base begin to rotate rather than continue along the linear path. For example, the loupe lens and the rotating base rotate from the procedure position to an inflection position as the WAVS continues to move along the linear path. The first repulsive magnetic force generated by the rotating and stationary magnets resists this rotation from the procedure position.

After passing the inflection position, in certain embodiments, the flux centerlines of the rotating and stationary magnets change position relative to each other, and the second repulsive magnetic force is generated by the rotating and stationary magnets. In certain embodiments, the second repulsive magnetic force causes the loupe lens and the rotating base to rotate away from the cornea to a safety position (also known as a second position). The second repulsive magnetic force then holds the rotating base against the stop on the body of the loupe lens turret, according to certain embodiments.

The inflection position may be expressed as a trigger, actuation, or inflection angle with respect to the flux centerline of the stationary magnet. For example, the inflection angle may be between 1 degree and 5 degrees, such as 2 degrees, 3 degrees, etc. Similarly, the second position may be expressed as a safety angle with respect to the flux centerline of the stationary magnet. For example, the safety angle may be between 30 degrees and 60 degrees, such as 45 degrees, etc.

Advantageously, the arm assembly may be tuned to actuate at a particular pressure or force against the cornea, such as between 5 grams and 15 grams, 10 grams, 15 grams, etc. The force may be dependent upon the magnetic strength of the stationary and rotating magnets, the separation distance between the repulsing sides of the stationary and rotating magnets in the procedure position, the off-center distance between the flux centerlines of the stationary and rotating magnets in the procedure position, etc.

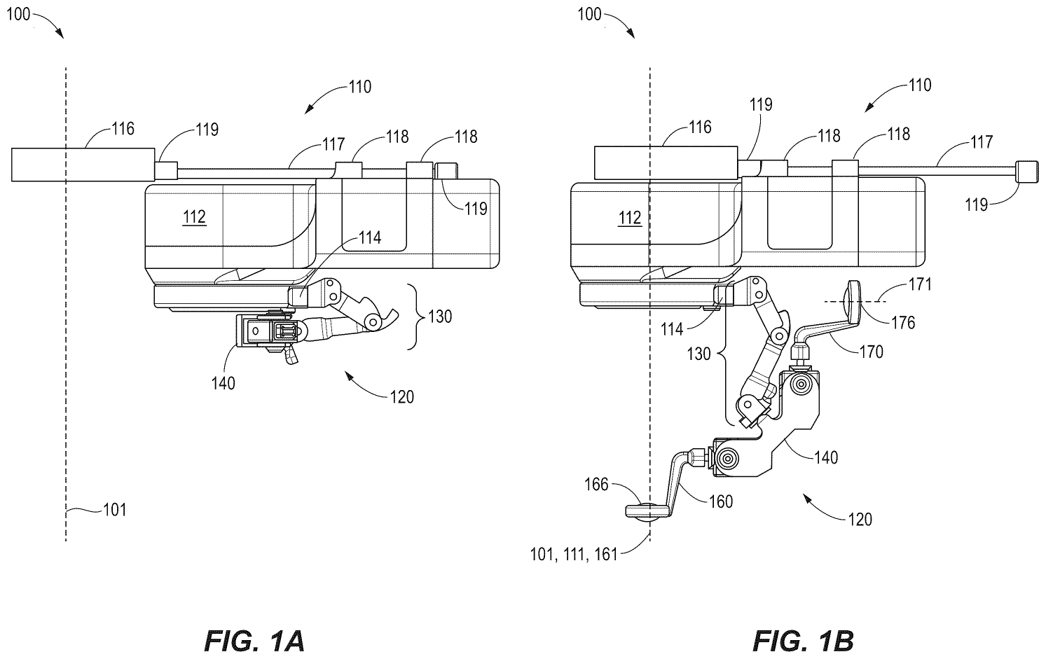

FIGS. 1A and 1B depict a left side view of a WAVS 100 in a stowed position and a deployed position (respectively), in accordance with embodiments of the present disclosure.

In certain embodiments, the WAVS 100 includes a reduction lens module 110 and a removable arm assembly 120. The reduction lens module 110 includes a body 112 that houses one or more reduction lenses, an arm assembly mount 114 that is configured to receive the arm assembly 120, and a microscope mount 116 that is configured to be attached to an ophthalmic microscope. The arm assembly mount 114 may be rotationally coupled to the body 112 to allow the surgeon to rotate the arm assembly 120 through 360° for convenience. Two rods 117 extend from the microscope mount 116, and two rod mounts 118 attach the body 112 to the rods 117. Each rod mount 118 may include a bearing, a bushing, etc., for each rod 117.

In certain embodiments, the surgeon (assistant, etc.) moves the body 112 of the reduction lens module 110 between the stowed and deployed positions by sliding the body 112 along the rods 117. The stops 119 prevent the body 112 from sliding off the rods 117 in the stowed and deployed positions. In some other embodiments, the rods 117 may be one or more linear rails, and the body 112 may include an electric motor with one or more drive gears that are coupled to the linear rails to move the reduction lens module 110 between the stowed and deployed positions in response to the actuation of a button, a switch, etc.

The arm assembly 120 includes an articulated arm 130 and a loupe lens turret 140. The loupe lens turret 140 is rotatably coupled to the articulated arm 130, and is configured to receive a removable loupe lens assembly 160 and a removable loupe lens assembly 170. The loupe lens assembly 160 and the loupe lens assembly 170 may be disposable or reusable (such as autoclavable, etc.).

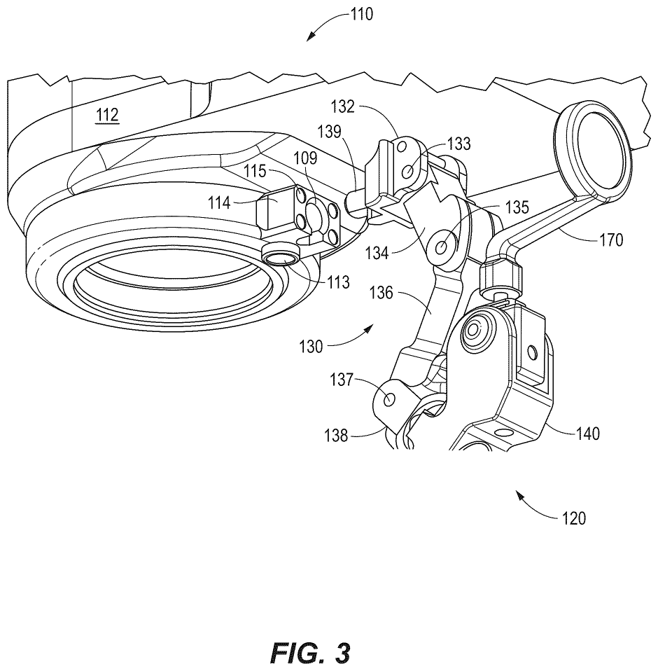

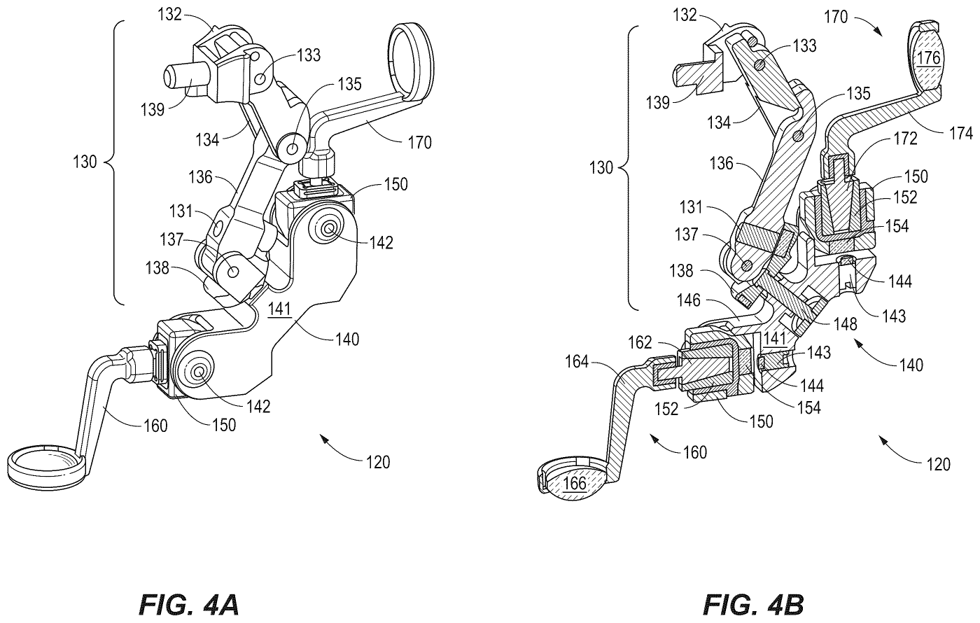

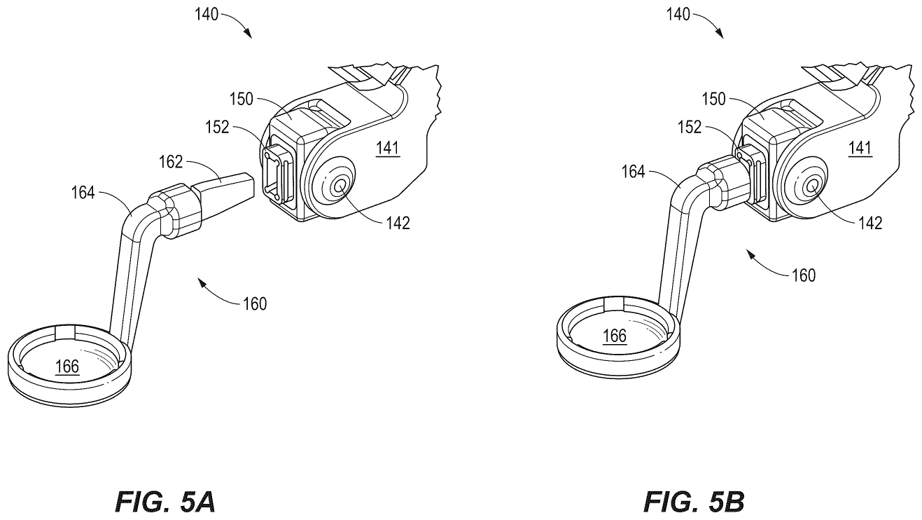

In the stowed position, the reduction lens within the body 112 of the reduction lens module 110 is not aligned with the optical axis 101 of the ophthalmic microscope, the articulated arm 130 is retracted, and the loupe lens turret 140 is disposed in a stowed orientation. The arm assembly 120 is held in in the stowed position by a magnetic attractive force generated by a magnet 113 located in the arm assembly mount 114 (not visible, see FIG. 3), which acts upon a magnetic material 131 located in the arm segment 136 (not visible, see FIGS. 4A, 4B). Generally, the loupe lens assemblies 160, 170 are not attached to the loupe lens turret 140 in the stowed position.

In the deployed position, the optical axis 111 of the reduction lens of the reduction lens module 110 is aligned with the optical axis 101 of the ophthalmic microscope, the articulated arm 130 is extended, the loupe lens turret 140 is disposed in either a first deployed orientation or a second deployed orientation, and the loupe lens assemblies 160, 170 are attached to the loupe lens turret 140. FIG. 1B depicts the loupe lens turret 140 disposed in the first orientation that aligns the optical axis 161 of the loupe lens 166 of the loupe lens assembly 160 with the optical axis 101 of the ophthalmic microscope. The loupe lens turret 140 may also be rotated 180° to the second deployed orientation to align the optical axis 171 of the loupe lens 176 of the loupe lens assembly 170 with the optical axis 101 of the ophthalmic microscope. The loupe lens turret 140 may be held in the first or second deployed orientations using a spring-loaded coupling (see FIG. 4B). The arm assembly 120 is held in the deployed position by gravity.

In certain embodiments, the loupe lenses 166, 176 may have different optical properties, such as different magnifications, different fields of view, etc. The surgeon may rotate the loupe lens turret 140 to align the optical axis 161, 171 of the desired loupe lens 166, 176 with the optical axis 101 of the ophthalmic microscope. In other embodiments, the loupe lenses 166, 176 may have the same optical properties, such as for redundancy, etc.

Generally, the reduction lens cooperates with the loupe lens 166, 176 to adjust a focal plane of the ophthalmic microscope from the cornea to the retina, to increase the magnification, and to increase the field of view. The reduction lens may be a singlet lens, a doublet lens, a triplet lens, etc. The loupe lens 166, 176 provides a fixed or variable wide field of view (or observation angle), magnification, etc. For example, different loupe lenses 166, 176 may provide different wide fields of view generally between 60° and 180°, such as 60°, 90°, 120°, etc., or a range of wide fields of view, such as 60° to 120°, 60° to 130°, etc. The working distance of the ophthalmic microscope may be adjusted by changing the characteristics of the arm assembly 120, such as a working distance of 175 mm, a working distance of 200 mm, etc.

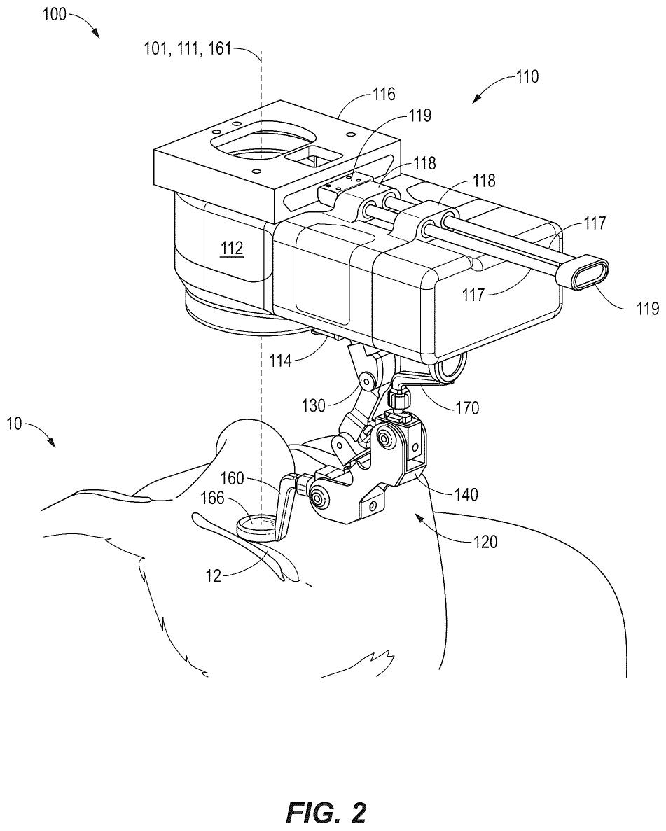

FIG. 2 depicts a perspective view of the WAVS 100 during a patient procedure, in accordance with embodiments of the present disclosure.

The WAVS 100 is disposed in the deployed position during the patient procedure. The optical axis 111 of the reduction lens and the optical axis 161 of the loupe lens 166 are aligned with the optical axis 101 of the ophthalmic microscope, and the loupe lens 166 is located proximate to the cornea 12 of the patient 10.

To prepare the WAVS 100 for the ophthalmic procedure, the surgeon may extend the arm assembly 120 from the stowed position to the deployed position, and attach the loupe lens assembly 160 to the loupe lens turret 140. If the loupe lens assembly 170 is anticipated to be used during the procedure, the loupe lens assembly 170 may also be attached to the loupe lens turret 140. In other words, only a single loupe lens assembly needs to be attached to the WAVS 100 during the procedure. The surgeon may then rotate the loupe lens turret 140 from the stowed orientation to the first deployed orientation, which aligns the optical axis 161 with the optical axis 111. The surgeon may then move the body 112 of the reduction lens module 110 along the rails 117 to the deployed position, which places the loupe lens 166 proximate to the cornea 12 of the patient 10, and aligns the optical axis 101 with the optical axes 111 and 161.

These steps may also be performed in a different sequence, such as moving the body 112 of the reduction lens module 110 along the rails 117 to the deployed position, extending the arm assembly 120 from the stowed position to the deployed position, and attaching the loupe lens assembly 160 to the loupe lens turret 140, and rotating the loupe lens turret 140 from the stowed orientation to the first deployed orientation.

During the focusing procedure, the height of the WAVS 100 above the patient 10 can be adjusted such that the distance between the lower surface of the loupe lens 166 and the cornea 12 is reduced or increased.

FIG. 3 depicts a perspective view of a portion of the reduction lens module 110 and a portion of the arm assembly 120 of the WAVS 100, in accordance with embodiments of the present disclosure. The portion of the arm assembly 120 is depicted in the deployed position and unattached to the reduction lens module 110.

In certain embodiments, the articulated arm 130 includes a reduction lens module mount 132, an arm segment 134, an arm segment 136, and a loupe lens turret mount 138. The reduction lens module mount 132 is rotatably coupled to the arm segment 134 via a pivot pin 133, the arm segment 134 is rotatably coupled to the arm segment 136 via a pivot pin 135, the arm segment 136 is rotatably coupled to the loupe lens turret mount 138 via a pivot pin 137, and the loupe lens turret mount 138 is rotatably coupled to the loupe lens turret 140 via a spring-loaded shaft 148 (see FIG. 4B).

In certain embodiments, the arm assembly mount 114 includes one or more magnets 115, and the reduction lens module mount 132 is made from magnetic material. The magnets 115 and the reduction lens module mount 132 are configured to magnetically couple the arm assembly 120 to the reduction lens module 110. In certain embodiments, the reduction lens module mount 132 may include a projection 139, and the arm assembly mount 114 may define a passage 109 that is configured to receive the projection 139. Other types of mounts may also be used, such as a bayonet mount, a clamp, a removable fastener, etc.

Advantageously, the arm assembly 120 may be configured to provide a particular working distance for the ophthalmic microscope, so that different arm assemblies 120 may be available before or during the procedure to provide different working distances. In the deployed position, the arm assembly 120 may extend over a vertical distance that contributes to the working distance for the ophthalmic microscope. The vertical distance may depend on certain characteristics of the arm assembly 120, such as the length of the arm segment 134, the length of the arm segment 136, the angle between the reduction lens module mount 132 and the arm segment 134 in the deployed positon, the angle between the arm segment 134 and the arm segment 136 in the deployed positon, and the angle between the arm segment 136 and the loupe lens turret mount 138 in the deployed positon, the length of the loupe lens turret 140 between the loupe lens turret mount 138 and the loupe lens assembly 160, the length of the loupe lens assembly 160, etc.

FIG. 4A depicts a perspective view of the arm assembly 120 of the WAVS 100, in accordance with embodiments of the present disclosure. The arm assembly 120 is depicted in the deployed position.

In the embodiments of FIG. 4A, the loupe lens turret 140 includes a body 141, a first rotating base 150 coupled to the body 141 via a first pivot joint 142, and a second rotating base 150 coupled to the body 141 via a second pivot joint 142. At least one loupe lens assembly 160, 170 is attached to the loupe lens turret 140 during the procedure. For example, the loupe lens assembly 160 may be attached to the first rotating base 150, and the loupe lens assembly 170 may be attached to the second rotating base 150. Alternatively, the loupe lens assembly 160 may be attached to the first rotating base 150, or the loupe lens assembly 170 may be attached to the second rotating base 150.

In certain other embodiments, the loupe lens turret 140 does not include the second rotating base 150 and the second pivot joint 142, and the arm assembly 120 does not include the loupe lens assembly 170. In other words, the loupe lens turret 140 is configured to receive one loupe lens assembly (such as the loupe lens assembly 160), and may be rotated between the stowed orientation and a deployed orientation (such as the first deployed orientation). In certain other embodiments, the loupe lens turret 140 may be rotated 180° from the deployed orientation to an additional stowed orientation. Advantageously, the loupe lens assemblies 160, 170 may be easily exchanged during the procedure if necessary.

FIG. 4B depicts a perspective sectional view of the arm assembly 120 depicted in FIG. 4A, in accordance with embodiments of the present disclosure. The arm assembly 120 is depicted in the deployed position.

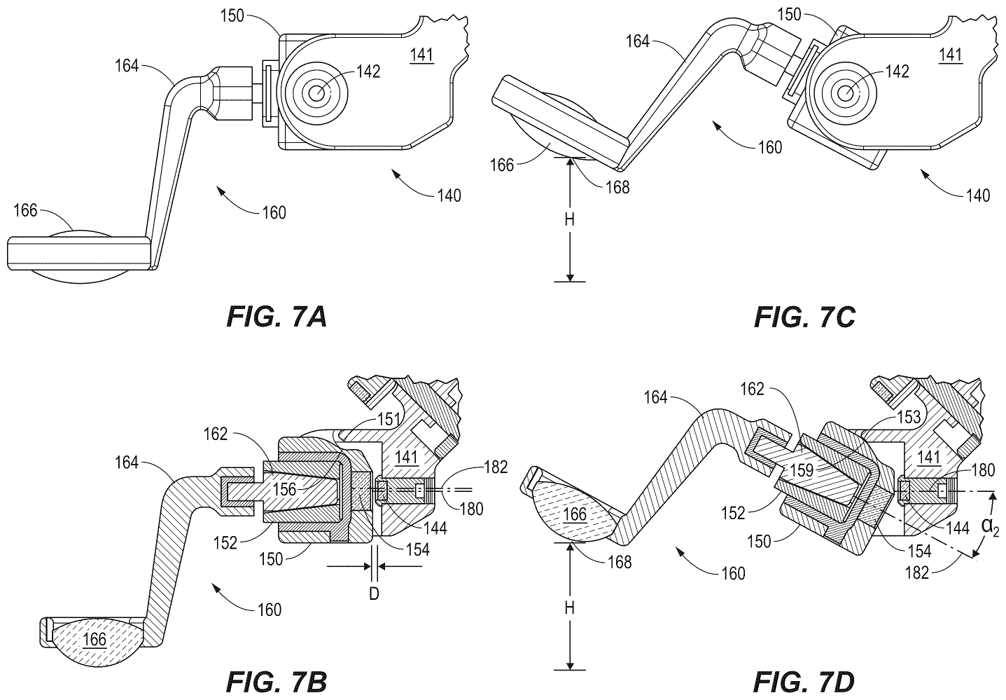

In the embodiments of FIG. 4B, the loupe lens turret 140 has a body 141 that includes a first stationary magnet 144 located proximate to the first rotating base 150, and a second stationary magnet 144 located proximate to the second rotating base 150. Each stationary magnet 144 has a repulsive side 145 and an attractive side 147 (see FIG. 6). The repulsive side 145 of the first stationary magnet 144 faces the first rotating base 150, and the repulsive side 145 of the second stationary magnet 144 faces the second rotating base 150.

The first rotating base 150 includes a socket 152 located at one end, and a first rotating magnet 154 located at the other end. Each rotating magnet 154 has a repulsive side 155 and an attractive side 157 (see FIG. 6). The repulsive side 155 of the first rotating magnet 154 faces the repulsive side 145 of the first stationary magnet 144, and the attractive side 157 of the first rotating magnet 154 faces the socket 152. Similarly, the repulsive side 155 of the second rotating magnet 154 faces the repulsive side 145 of the second stationary magnet 144, and the attractive side 157 of the second rotating magnet 154 faces the socket 152. The repulsive sides 155 of the rotating magnets 154 may be covered with an epoxy seal.

In certain embodiments, each stationary magnet 144 may be mounted to an adjustable segment, such as a threaded insert 143 that is movable within a threaded passage 149 (see FIG. 6). Advantageously, the adjustable segment may be moved closer to and farther away from the rotating base 150, which moves the stationary magnet 144 closer to and farther away from the rotating magnet 154. In certain other embodiments, the threaded insert 143 may be made from magnetic steel (such as 400 series stainless steel), and the stationary magnet 144 is not mounted therein. The rotating magnet 154 and the magnetic threaded insert 143 generate the first and second repulsive magnetic forces. In some other embodiments, the rotating magnet 154 may be replaced by a magnetic insert made from magnetic steel (such as 400 series stainless steel), and the stationary magnet 144 and the magnetic insert generate the first and second repulsive magnetic forces.

Other methods may be used to adjust the distance between the stationary magnet 144 and the rotating magnet 154. In certain embodiments, the adjustable segment may be a cylinder that forms an interference fit within a cylindrical passage, etc. In certain other embodiments, the adjustable segment may be a cylinder that has a pin that engages a slot with periodic cutouts that extends along the passageway, etc. In certain other embodiments, the adjustable segment may be a set of segments, and each segment includes a stationary magnet 144 and has a different length.

The loupe lens assembly 160 includes an alignment tab 162, a shaft 164, and the loupe lens 166. Similarly, the loupe lens assembly 170 includes an alignment tab 172, a shaft 174, and the loupe lens 176. The socket 152 of the first rotating base 150 is configured to receive the alignment tab 162 of the loupe lens assembly 160, and the socket 152 of the second rotating base 150 is configured to receive the alignment tab 172 of the loupe lens assembly 170. In certain embodiments, the socket 152 may be secured within the rotating base 150 using potting compound, adhesive, silicone, etc. Similarly, the alignment tab 162, 172 may be secured with the shaft 164, 174 using potting compound, adhesive, silicone, etc.

In certain embodiments, the alignment tab 162 is made from magnetic material, and is held in the socket 152 of the first rotating base 150 by a magnetic attractive force generated by the first rotating magnet 154 of the first rotating base 150. Similarly, the alignment tab 172 is made from magnetic material, and is held in the socket 152 of the second rotating base 150 by a magnetic attractive force generated by the first rotating magnet 154 of the first rotating base 150.

Other methods may be used to secure the loupe lens assembly 160 to the first rotating base 150, and to secure the loupe lens assembly 170 to the second rotating base 150. In certain embodiments, the alignment tab 162, 172 may be held within the socket 152 by an interference fit (also known as a press fit or a friction fit), a detachable snap fit, etc. In certain other embodiments, the loupe lens assembly 160, 170 may be secured to the rotating base 150 using a fastener, such as a captive screw, a spring-loaded plunger, a swell latch, a quick release pin, etc. In certain other embodiments, the loupe lens assembly 160, 170 may be secured to the rotating base 150 using a bayonet mount, a clamp, etc.

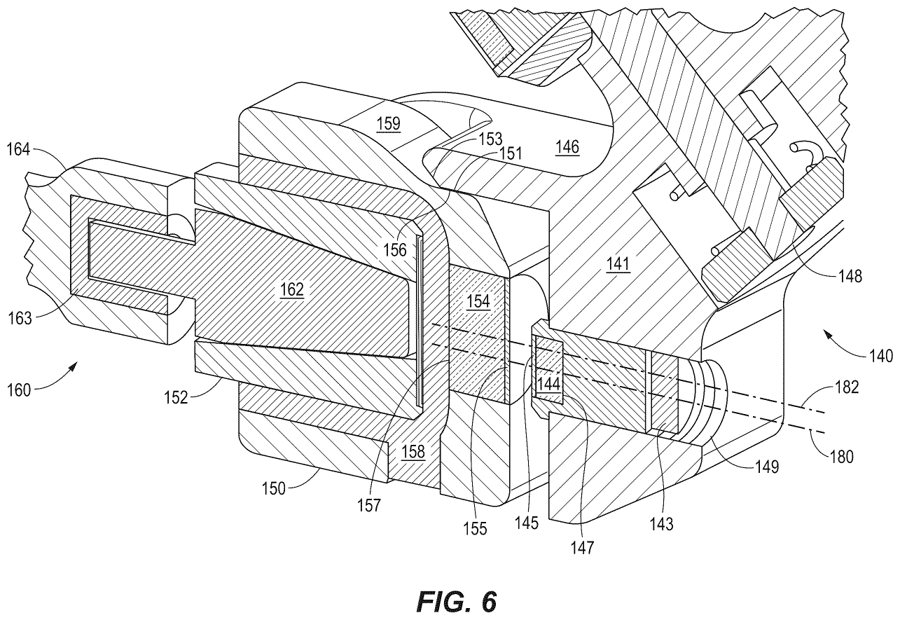

FIGS. 5A and 5B depict perspective views of a portion of a loupe lens turret 140 and a loupe lens assembly 160 depicted in FIG. 4A, in accordance with embodiments of the present disclosure.

FIG. 5A depicts the loupe lens assembly 160 unattached to the socket 152 of the first rotating base 150.

FIG. 5B depicts the loupe lens assembly 160 attached to the socket 152 of the first rotating base 150.

FIG. 6 depicts a perspective sectional view of a portion of the loupe lens turret 140 and a portion of the loupe lens assembly 160 depicted in FIG. 4A, in accordance with embodiments of the present disclosure. The loupe lens turret 140 is depicted in the procedure position and the first orientation.

The first stationary magnet 144 has a flux centerline 180, and the first rotating magnet 154 has a flux centerline 182. The body 141 has a stop 146 that has a first stop surface 151 and a second stop surface 153, and the rotating base 150 has a first stop surface 156 and a second stop surface 159.

The flux centerline 182 of the first rotating magnet 154 is situated parallel to and offset from the flux centerline 180 of the stationary magnet 144, which generates the first repulsive magnetic force that holds the first stop surface 156 of the rotating base 150 against the first stop surface 151 of the stop 146 in the procedure position.

In order the ensure that the optical axis 161 is aligned to the optical axis 111 when the loupe lens assembly 160 is disposed in the procedure position, the socket 152 may be aligned and then secured within the first rotating base 150, and the alignment tab 162 may be aligned and then secured within the shaft 164. In certain embodiments, the potting compound 158 secures the socket 152 within the first rotating base 150 after alignment, and the potting compound 163 secures the alignment tab 162 within the shaft 164 after alignment. Advantageously, the potting compound 158 may be magnetically transparent to the attractive magnetic force that secures the alignment tab 162 in the socket 152.

Similarly, in order the ensure that the optical axis 171 is aligned to the optical axis 111 when the loupe lens assembly 170 is disposed in the procedure position, the socket 152 may aligned and then secured within the second rotating base 150, and the alignment tab 172 may be aligned and then secured within the shaft 174. In certain embodiments, the potting compound 158 secures the socket 152 within the second rotating base 150 after alignment, and the potting compound 163 secures the alignment tab 162 within the shaft 164 after alignment. Advantageously, the potting compound 158 may be magnetically transparent to the attractive magnetic force that secures the alignment tab 172 in the socket 152.

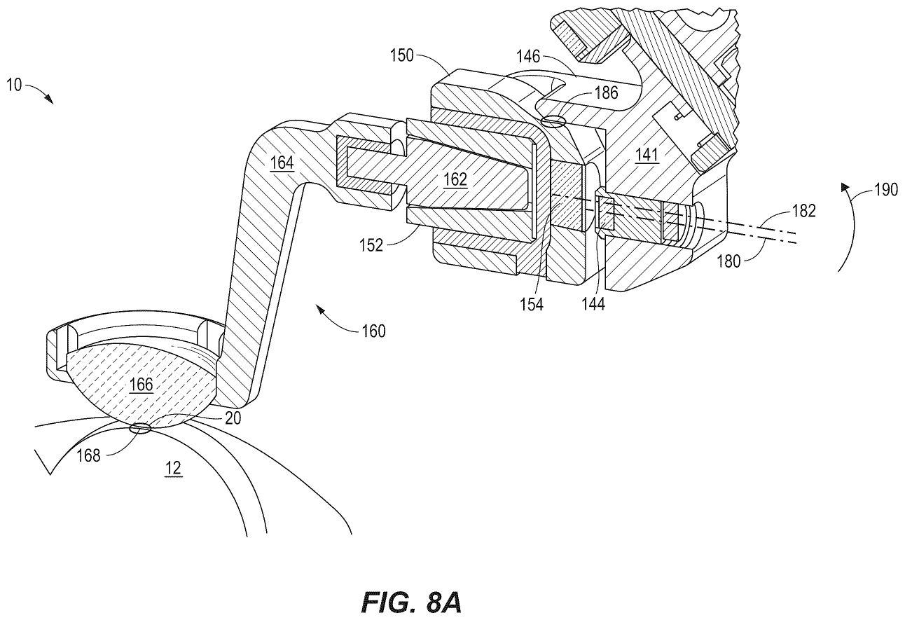

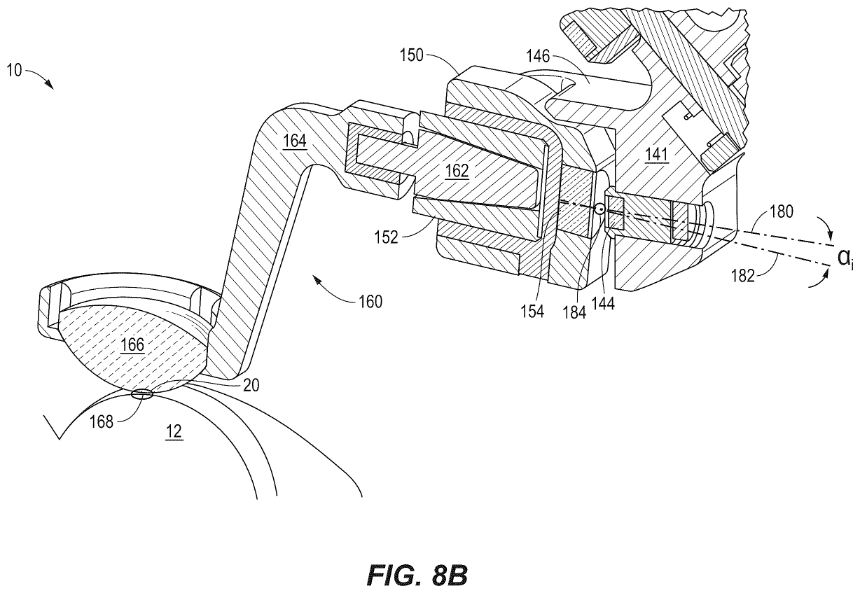

FIGS. 7A and 7B depict a left side view and a left side sectional view (respectively) of a portion of the loupe lens turret 140 and the loupe lens assembly 160 in the procedure position, in accordance with embodiments of the present disclosure. The loupe lens turret 140 and the loupe lens assembly 160 are depicted in the first orientation.

The flux centerline 182 of the first rotating magnet 154 is situated parallel to and offset from the flux centerline 180 of the stationary magnet 144 in the procedure position, which generates the first repulsive magnetic force that holds the first stop surface 156 of the rotating base 150 against the first stop surface 151 of the stop 146 in the procedure position.

A distance “D” separates the repulsive side 145 of the first stationary magnet 144 and the repulsive side 155 of the first rotating magnet 154 in the procedure position. The separation distance D may be increased or decreased (e.g., controlled) by adjusting the position of the threaded insert 143 within the threaded passage 149, which decreases or increases (respectively) the magnitudes of the first and second magnetic repulsive forces generated by the first stationary magnet 144 and the first rotating magnet 154. For example, the distance D may be adjusted between a minimum distance (such as 5 mm, etc.) and a maximum distance (such as 20 mm, etc.). The minimum distance may be dependent upon the clearance required by the first rotating base 150, and the maximum distance may be dependent upon the length of the threaded passage 149.

FIGS. 7C and 7D depict a left side view and a left side sectional view (respectively) of a portion of the loupe lens turret 140 and the loupe lens assembly 160 in the safety position, in accordance with embodiments of the present disclosure. The loupe lens turret 140 and the loupe lens assembly 160 are depicted in the first orientation.

The flux centerline 182 of the first rotating magnet 154 has rotated away from the flux centerline 180 of the stationary magnet 144 and forms an angle α₂ with respect thereto, which generates the second repulsive magnetic force that holds the second stop surface 159 of the rotating base 150 against the second stop surface 153 of the stop 146.

A distance “H” represents the distance between the cornea 12 and the lower surface 168 of the loupe lens 166 in the safety position. The distance H is generally dependent on the angle α₂ and the length of the shaft 164, and the angle α₂ is generally dependent on the clearance required by the first rotating base 150, and the locations of the second stop surface 153 and the second stop surface 159. For example, the distance H may be between a minimum distance (such as X mm) and a maximum distance (such as Y mm), such as Z mm, and the angle α₂ may be between 30 degrees and 60 degrees, such as 45 degrees, etc.

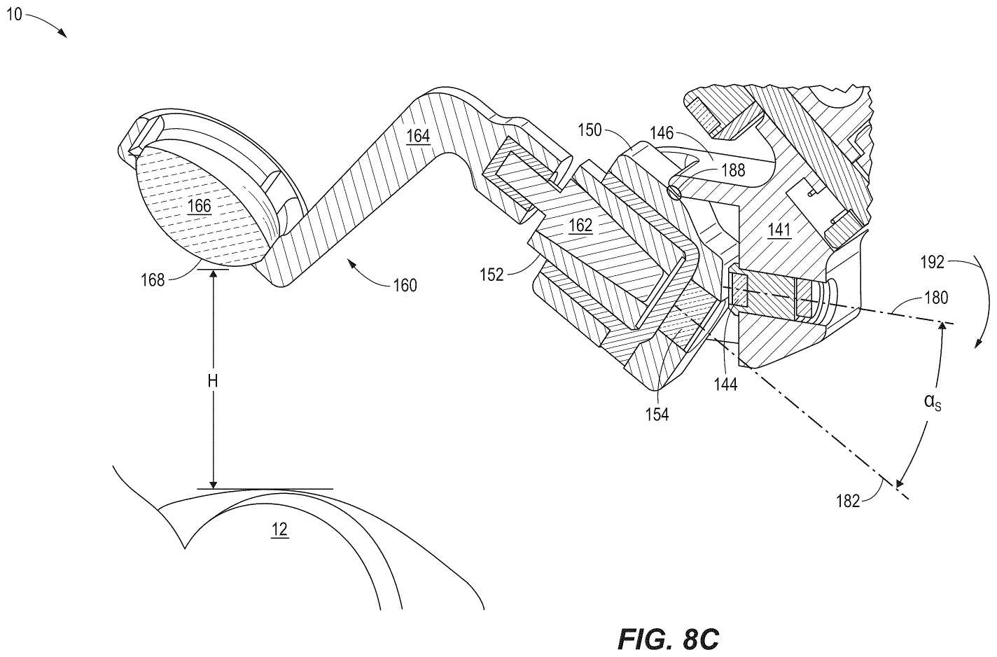

FIGS. 8A, 8B, 8C depict perspective sectional views of a portion of the loupe lens turret 140 and the loupe lens assembly 160 in the procedure position, the inflection position, and the safety position (respectively), in accordance with embodiments of the present disclosure. The loupe lens turret 140 and the loupe lens assembly 160 are depicted in the first orientation.

FIG. 8A depicts a perspective sectional view of a portion of the loupe lens turret 140 and the loupe lens assembly 160 in the procedure position, in accordance with embodiments of the present disclosure. The loupe lens turret 140 and the loupe lens assembly 160 are depicted in the first orientation.

The focusing procedure has caused the loupe lens 166 to contact the cornea 12.

The flux centerline 182 of the first rotating magnet 154 is situated parallel to and offset from the flux centerline 180 of the stationary magnet 144, which generates the first repulsive magnetic force 190 that rotates the rotating base 150 (in a first direction), and holds the rotating base 150 against the stop 146 in the procedure position, as identified by the contact area 186.

The lower surface 168 of the loupe lens 166 is in contact with the cornea 12, as identified by contact area 20, and initially applies a contact force between 5 grams and 15 grams to the cornea 12, such as about 10 grams.

FIG. 8B depicts a perspective sectional view of a portion of the loupe lens turret 140 and the loupe lens assembly 160 in the inflection position, in accordance with embodiments of the present disclosure. The loupe lens turret 140 and the loupe lens assembly 160 are depicted in the first orientation.

The focusing procedure caused the loupe lens 166 to remain in contact with the cornea 12, and caused the first rotating base 150 to rotate (in a second direction) away from the procedure position to the inflection position.

In the inflection position, the flux centerline 182 of the first rotating magnet 154 forms an inflection angle αᵢ with respect to the flux centerline 180 of the first stationary magnet 144, and the flux centerlines 180 and 182 intersect at inflection point 184. While the first rotating magnet 154 and the first stationary magnet 144 generate the largest repulsive magnetic force at the inflection position, this repulsive force does not cause the first rotating base 150 to rotate in either the first direction or the second direction, and the lower surface 168 of the loupe lens 166 rests on the cornea 12.

The distance D, the offset distance between flux centerline 180 and flux centerline 182 in the procedure position, and the repulsive magnetic forces that are generated between stationary magnet 144 and rotating magnet 154 may determine the inflection angle αᵢ and the force needed to reach that angle. As discussed above, in certain embodiments, the distance D may be adjustable by mounting the stationary magnet 144 to an adjustable segment, such as a threaded insert 143 that is movable within a threaded passage 149. This axial degree of freedom for the stationary magnet 144 allows a certain amount of tuning by the end user. In other embodiments, the stationary magnet 144 may be fixed within the body 141 at a predetermined distance D, the offset distance may be set to a predetermined value, and the repulsive magnetic force may be set to a predetermined value such that the inflection angle αᵢ and the force needed to reach that angle are non-adjustable by the end user.

FIG. 8C depicts a perspective sectional view of a portion of the loupe lens turret 140 and the loupe lens assembly 160 in the safety position, in accordance with embodiments of the present disclosure. The loupe lens turret 140 and the loupe lens assembly 160 are depicted in the first orientation.

After the focusing procedure causes the flux centerline 182 to rotate past the inflection point 184 (in the second direction), the first rotating magnet 154 and the first stationary magnet 144 begin to generate the second repulsive magnetic force 192, which begins to rotate the first rotating base 150 toward the safety position and move the loupe lens 166 away from the cornea 12. When the second stop surface 159 contacts the second stop surface 153, the rotation stops, and the second repulsive magnetic force 192 holds the rotating base 150 against the stop 146 in the safety position, as identified by the contact area 188.

The flux centerline 182 of the first rotating magnet 154 forms a safety angle as with respect to the flux centerline 180 of the first stationary magnet 144, and the lower surface 168 of the loupe lens 166 is located at the distance H from the cornea 12.

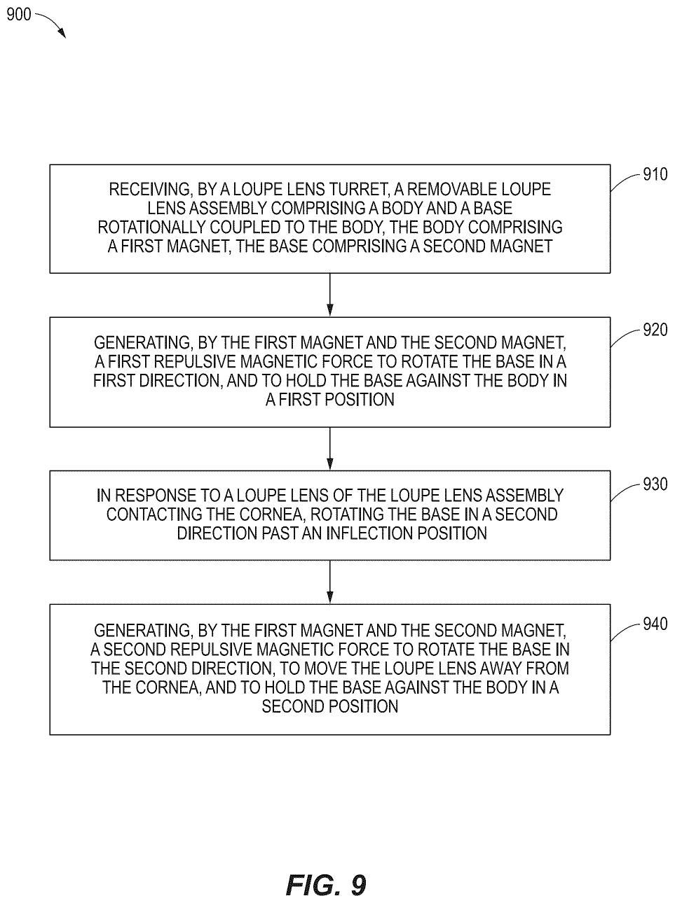

FIG. 9 depicts an example process flow diagram 900 presenting functionality associated with the arm assembly 120 of the example WAVS 100 during a procedure, in accordance with certain embodiments of the present disclosure.

At 910, a removable loupe lens assembly 160 is received by a loupe lens turret 140. The loupe lens turret 140 comprises a body 141 and a base 150 that is rotationally coupled to the body 141. The body 141 comprises a first magnet 144, and the base 150 comprises a second magnet 154.

At 920, a first repulsive magnetic force 190 is generated by the first magnet 144 and the second magnet 154 to rotate the base 150 in a first direction, and to hold the base 150 against the body 141 in a first position (such as the procedure position).

At 930, in response to the loupe lens 166 contacting the cornea 12, the base 150 is rotated in a second direction past an inflection position. In the inflection position, no repulsive magnetic force is generated by the first magnet and the second magnet.

At 940, a second repulsive magnetic force 192 is generated by the first magnet 144 and the second magnet 154 to rotate the base 150 in the second direction, to move the loupe lens 166 away from the cornea 12, and to hold the base 150 against the body 141 in a second position (such as the safety position).

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. An arm assembly for a wide angle viewing system, comprising:
an articulated arm configured to be coupled to a reduction lens module; and
a loupe lens turret coupled to the articulated arm, the loupe lens turret comprising:
a body comprising a first magnet, and
a base rotationally coupled to the body and configured to receive a removable loupe lens assembly, the base comprising a second magnet, wherein the first magnet and the second magnet are configured to generate:
a first repulsive magnetic force to rotate the base in a first direction and hold the base against the body in a first position, and
a second repulsive magnetic force to rotate the base in a second direction and hold the base against the body in a second position.

2. The arm assembly of claim 1, wherein:
the removable loupe lens assembly comprises a loupe lens and a magnetic alignment tab;
the base further comprises a socket to receive the magnetic alignment tab; and
an attracting side of the second magnet is configured to hold the magnetic alignment tab in the socket.

3. The arm assembly of claim 2, wherein:
the body further comprises a stop and a pivot joint coupled to the base;
when the loupe lens contacts a cornea, the base is rotated about the pivot joint in the second direction past an inflection position; and
the second repulsive magnetic force rotates the base about the pivot joint in the second direction, moves the loupe lens away from the cornea, and holds the base against the stop in the second position.

4. The arm assembly of claim 3, wherein, in the inflection position, the first repulsive magnetic force is not generated by the first magnet and the second magnet, and the second repulsive magnetic force is not generated by the first magnet and the second magnet.

5. The arm assembly of claim 4, wherein:
the inflection position is a rotation of the base in the second direction of between 1 degree and 5 degrees from the first position; and
the second position is a rotation of the base in the second direction of between 30 degrees and 60 degrees from the first position.

6. The arm assembly of claim 5, wherein, in the second position, a lower surface of the loupe lens is located at least a minimum distance above the cornea.

7. The arm assembly of claim 6, wherein the minimum distance is at least 5 mm.

8. The arm assembly of claim 1, wherein:
the first magnet has a first flux centerline, a first attracting side, and a first repulsing side;
the second magnet has a second flux centerline, a second attractive side, and a second repulsing side; and
the first repulsing side faces the second repulsing side.

9. The arm assembly of claim 8, wherein:
when the base is disposed in the first position, the second flux centerline is parallel to, and offset from, the first flux centerline; and
when the base is disposed in the second position, the second flux centerline is not parallel to the first flux centerline.

10. The arm assembly of claim 9, wherein:
the second magnet is adjustable along the second flux centerline to control a separation distance between the first magnet and the second magnet when the base is disposed in the first position; and
the separation distance determines, at least in part, an inflection position at which no repulsive magnetic force is generated by the first magnet and the second magnet.

11. The arm assembly of claim 10, wherein, in the first position, the first repulsing side is separated from the second repulsing side by the separation distance.

12. The arm assembly of claim 11, wherein the separation distance is at least 5 mm.

13. The arm assembly of claim 10, wherein:
the inflection position is a rotation of the base in the second direction of between 1 degree and 5 degrees from the first position; and
the second position is a rotation of the base in the second direction of between 30 degrees and 60 degrees from the first position.

14. The arm assembly of claim 1, wherein the articulated arm comprises:
a first arm segment comprising a mount configured to be removably coupled to the reduction lens module; and
a second arm segment rotationally coupled to the first arm segment and rotationally coupled to the loupe lens turret.

15. A method for operating an arm assembly for a wide angle viewing system, comprising:
receiving, by a loupe lens turret, a removable loupe lens assembly, the loupe lens turret comprising a body and a base rotationally coupled to the body, the body comprising a first magnet, the base comprising a second magnet;
generating, by the first magnet and the second magnet, a first repulsive magnetic force to rotate the base in a first direction, and to hold the base against the body in a first position;
in response to a loupe lens of the removable loupe lens assembly contacting a cornea of a patient, rotating the base in a second direction past an inflection position; and
generating, by the first magnet and the second magnet, a second repulsive magnetic force to rotate the base in the second direction, to move the loupe lens away from the cornea, and to hold the base against the body in a second position.

16. The method of claim 15, wherein, in the inflection position, the first repulsive magnetic force is not generated by the first magnet and the second magnet, and the second repulsive magnetic force is not generated by the first magnet and the second magnet.

17. The method of claim 15, wherein:
the removable loupe lens assembly comprises the loupe lens and a magnetic alignment tab;
the base further comprises a socket to receive the magnetic alignment tab; and
an attracting side of the second magnet is configured to hold the magnetic alignment tab in the socket.

18. The method of claim 15, wherein:
the first magnet has a first flux centerline, a first attracting side, and a first repulsing side;
the second magnet has a second flux centerline, a second attractive side, and a second repulsing side;
the first repulsing side faces the second repulsing side;
when the base is disposed in the first position, the first flux centerline is parallel to, and offset from, the second flux centerline which generates the first repulsive magnetic force; and
when the base is disposed in the second position, the first flux centerline is not parallel to the second flux centerline which generates the second repulsive magnetic force.

19. The method of claim 18, further comprising:
adjusting the second magnet along the second flux centerline to control a distance between the first magnet and the second magnet when the base is disposed in the first position,
wherein the distance determines, at least in part, the inflection position.

20. The method of claim 15, wherein:
the inflection position is a rotation of the base in the second direction of between 1 degree and 5 degrees from the first position; and
the second position is a rotation of the base in the second direction of between 30 degrees and 60 degrees from the first position.
